(19) United States
(12) Patent Application Publication
(10) Pub. No.: US 2021/0379106 A1
(43) Pub. Date: Dec. 9, 2021

(54) OXABICYCLOHEPTANES FOR ENHANCING CAR T CELL FUNCTION

(71) Applicants: Lixte Biotechnology, Inc., East Setauket, NY (US); City of Hope, Duarte, CA (US)

(72) Inventors: John S. KOVACH, East Setauket, NY (US); Stephen J. FORMAN, San Marino, CA (US); Xiuli WANG, Temple City, CA (US)

(21) Appl. No.: 17/252,160

(22) PCT Filed: Jun. 13, 2019
(86) PCT No.: PCT/US2019/037015

§ 371 Date: Dec. 14, 2020

Related U.S. Application Data
(60) Provisional application No. 62/685,132, filed on Jun. 14, 2018.

(51) Int. Cl. A61K 35/17; A61K 31/33; C12N 5/0783; A61P 35/02

(52) U.S. Cl. CPC A61K 35/17; A61P 35/02; C12N 5/0636; A61K 31/33

(57) ABSTRACT

The present invention provides a method of enhancing the function of CAR T cells comprising administering to the CAR T cells a PP2A inhibitor and optionally one or more anti-cancer therapies.

### CROSS-REFERENCE TO RELATED APPLICATIONS

The present application is a § 371 National Stage of PCT International Application No. PCT/US2019/037015, filed Jun. 13, 2019, which claims the benefit of U.S. Provisional Patent Application No. 62/685,132, filed Jun. 14, 2018, and the entirety of each are hereby incorporated by reference herein.

### SEQUENCE LISTING

The instant application contains a Sequence Listing which has been submitted in ASCII format and is hereby incorporated by reference in its entirety. The ASCII copy, created on Jul. 17, 2019, is named “Sequence Listing_SL_ST25.txt” and is 3 kilobytes in size.

### BACKGROUND OF THE INVENTION

Protein phosphatase 2A (PP2A) is a ubiquitous serine/threonine phosphatase that dephosphorylates numerous proteins of both ATM/ATR-dependent and -independent response pathways (Mumby, M. 2007). Pharmacologic inhibition of PP2A has previously been shown to sensitize cancer cells to radiation-mediated DNA damage via constitutive phosphorylation of various signaling proteins, such as p53, γH2AX, PLK1 and Akt, resulting in cell cycle deregulation, inhibition of DNA repair, and apoptosis (Wei, D. et al. 2013).

Cantharidin, the principle active ingredient of blister beetle extract (Mylabris), is a compound derived from traditional Chinese medicine that has been shown to be a potent inhibitor of PP2A (Efferth, T. et al. 2005). Although cantharidin has previously been used in the treatment of hepatomas and has shown efficacy against multidrug-resistant leukemia cell lines (Efferth, T. et al. 2002), its severe toxicity limits its clinical usefulness. Cantharidin, a naturally occurring toxin, and its demethylated analog, norcantharidin, both potent inhibitorsof PP2A (Bertini et al. 2009) were reported to ha anti-cancer activity in patients in China with gastrointestinal cancers (Wang et al. 1989) although little clinical detail is available.

Fostriecin, another selective inhibitor of PP2A was evaluated in several US NCI-sponsored phase 1 trials over twenty years ago. In the largest trial, fostriecin was associated with disease stability in 16 (34.8%) of 46 solid tumor patients without dose-limiting toxicity (DLT) (Lê et al. 2004). No trials were completed because of insufficient drug supply.

LB100 is a small molecule derivative of cantharidin with significantly less toxicity. LB-100 and its lipid-soluble homolog, LB-102, inhibit proliferation of cell lines from a variety of human solid tumors. Both compounds potentiate the activity without significantly increasing the toxicity of cisplatin, doxorubicin, and temozolomide against xenografts of pancreatic and hepatocellular carcinoma; fibrosarcoma; pheochromocytoma; neuroblastoma; and glioblastoma and of focal X-ray against pancreatic, nasopharyngeal and glioblastoma xenografts (Bai et al., 2014a; Bai et al., 2014b; Zhang et al., 2010; Matiniova et al., 2011; Lu et al., 2009; Wei et al., 2013; Lv et al., 2014; Gordon et al., 2015). In addition, LB-100 reversed resistance to cisplatin in ovarian carcinoma and medulloblastoma xenografts (Chang et al., 2015; Ho et al., 2016). Previous pre-clinical studies have shown that LB100 can enhance the cytotoxic effects of temozolomide, doxorubicin, and radiation therapy against glioblastoma (GBM), metastatic pheochromocytoma, and pancreatic cancer (Wei, D. et al. 2013; Lu, J. et al. 2009; Zhang, C. et al. 2010; Martiniova, L. et al. 2011). LB100 is also undergoing a phase 1 study in combination with docetaxel for the treatment of solid tumors (Chung, V. 2013).

### BRIEF DESCRIPTION OF THE DRAWINGS

FIG. 1. Generation of CD19 CAR T cells PBMC from healthy donor were activated with CD3/CD28 beads and transduced with lentivirus encoding second generation of CD19CAR containing costimulatory signaling CD28. After 14 days of ex vivo expansion, percentages of CD8 and CAR positive T cells were determined by flow cytometry.

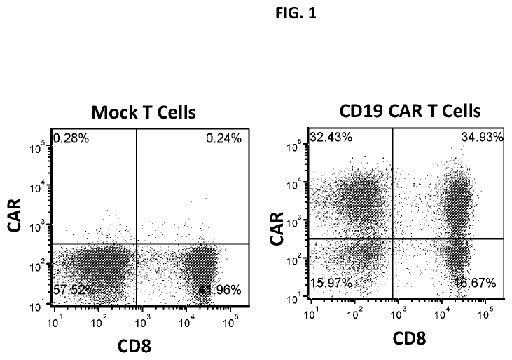

FIG. 2. LB100 improves effector function of CD19CAR T cells in a dose dependent manner Propagated CD19 CAR T cells were re-stimulated with CD19 antibody coated beads and concomitantly treated with different concentrations of LB100 as indicated on days 1, 3 and 5. Post treatment, 0.1×10⁶ CD19 CART cells were then co-cultured with 0.1×10⁶ different CD19+ tumor cells for 24 hours followed by surface staining of CAR and CD8 and intracellular staining of IFN-γ. Percentages of IFNγ+CD8+ CAR T cells in gated CAR positive population are presented.

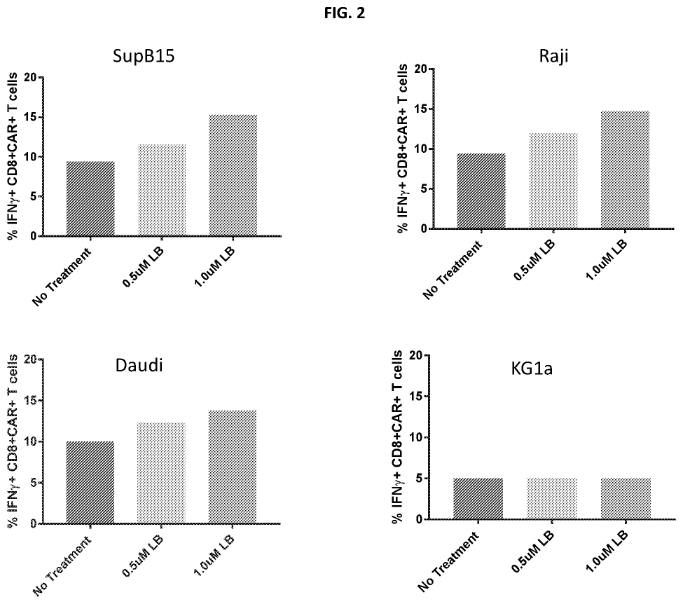

FIG. 3. LB100 and anti-PD-1 synergistically improves effector function of CD19CAR T cells Propagated CD19 CAR T cells were re-stimulated with CD3/CD28 beads and concomitantly treated as indicated on days 1, 3 and 5. Post treatment, 0.1×10⁶ CD19CAR T cells were then co-cultured with 0.1×10⁶ CD19+Daudi lymphoma cells for 24 hours followed by surface staining of CAR and CD8 and intracellular staining of IFN-γ. Percentages of IFN-γ+CD8+ CAR T cells (A) and FACS plots (B) in gated CAR positive population are presented.

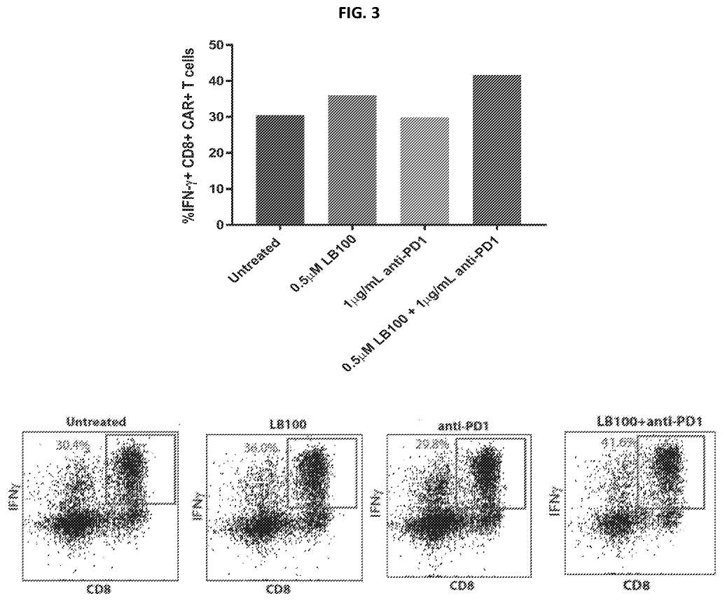

FIG. 4. LB100 and anti-PD-1 synergistically improves effector function of CD19CAR T cells Propagated CD19CAR T cells were re-stimulated with CD19 coated beads and concomitantly treated as indicated on days 1, 3 and 5. Post treatment, 0.1×10⁶ CD19CAR T cells were then co-cultured with 0.1×10⁶ different CD19+ tumor cells for 24 hours followed by surface staining of CAR and CD8 and intracellular staining of IFN-γ. Myeloid leukemic cells (KG1a) were used as negative control. Percentages of IFN-γ+CD8+ CAR T cells in gated CAR positive population are presented.

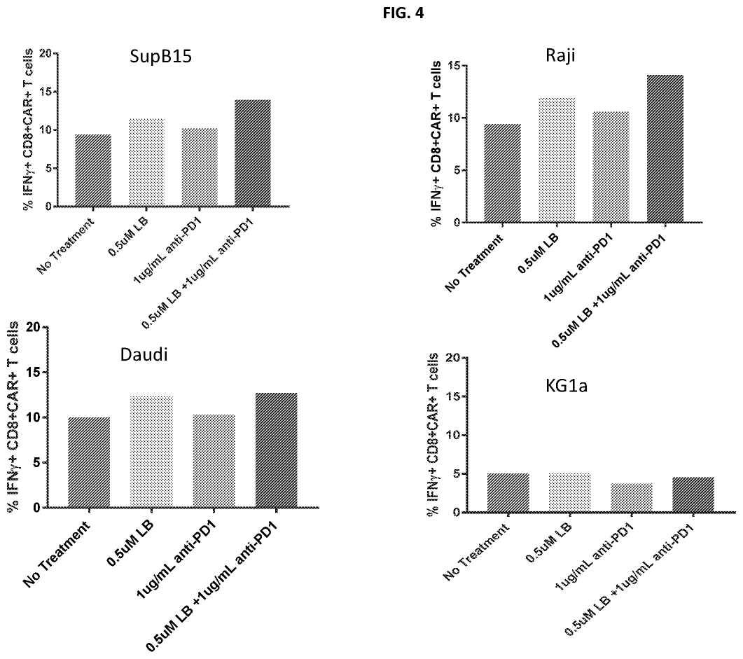

FIG. 5. LB100 and anti-PD-1 synergistically enhance antitumor activity of CD19CAR T cells in NSG mouse model (A) 0.5×10⁶ acute lymphoid leukemic SupB15 cells engineered with GFP firefly luciferase (GFPffluc+) were intravenously (i.v) inoculated into NSG mice on day −5. After confirmation of tumor engraftment, 1×10⁶ expanded CD19CAR T cells were adoptively transferred (i.v) into tumor-bearing mice. Mice were treated intraperitoneally with LB100 (4 μg/mouse), anti-PD-1 (Nivolumab) (200 μg/mouse) or LB100+Nivolumab every other day as combination. Tumor signals were monitored by biophotonic imaging. (B) Kaplan-Meier survival curve. N=5 mice per group.

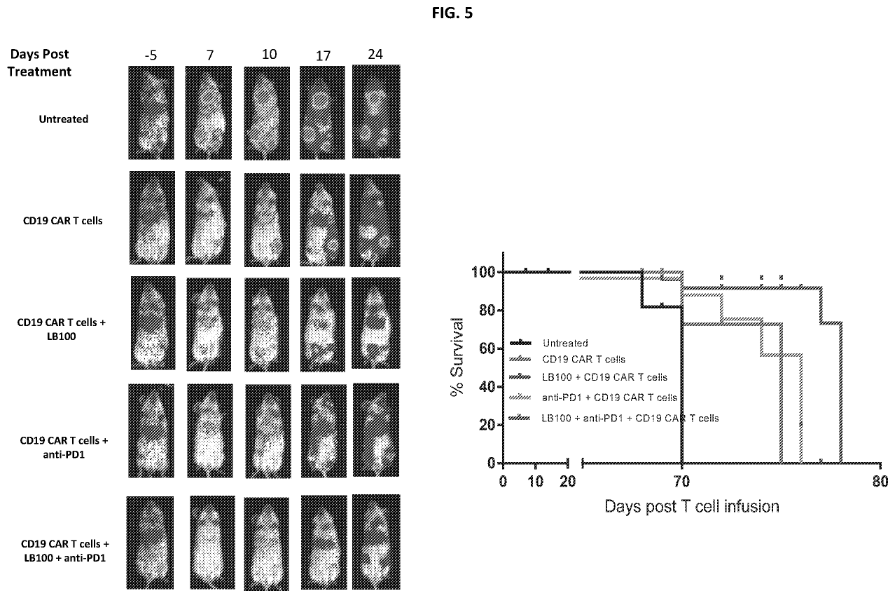

### DETAILED DESCRIPTION OF THE INVENTION

The present invention provides, inter alia, a method of enhancing the function of chimeric antigen receptor (CAR) T cells comprising administering to the CAR T cells a PP2A inhibitor so as to thereby enhance the function of the CAR T cells. In some embodiments, a method of enhancing the function of CAR T cells comprises enhancing and/or promoting their production of interferon gamma.

Chimeric antigen receptors (CAR) generally include a scFv targeted to an antigen of interest (e.g., CD19), a spacer region, a transmembrane domain, a costimulatory domain and a CD3zeta domain. Suitable spacer regions include, e.g., all or part of an immunoglobulin (e.g., IgG1, IgG2, IgG3, IgG4) hinge region, i.e., the sequence that falls between the CH1 and CH2 domains of an immunoglobulin, e.g., an IgG4 Fc hinge or a CD8 hinge. Some spacer regions include an immunoglobulin CH3 domain or both a CH3 domain and a CH2 domain.

A variety of transmembrane domains can be used, including the human CD28 transmembrane domain, the human CD8 transmembrane domain, and the human CD4 transmembrane domain. The costimulatory domain can be any domain that is suitable for use with a CD3ζ signaling domain. In some cases, the costimulatory domain is a CD28 costimulatory domain that includes a sequence that is at least 90%, at least 95%, at least 98% identical to or identical to: RSKRSRLLHSDYIVINMTPRRPGPTRKHYQPYAPPRDFAAYRS (SEQ ID NO:1) or RSKRSRGGHSDYIVINMTPRRPGPTRKHYQPYAPPRDFAAYRS (SEQ ID NO:2).

In some cases the co-signaling domain is a 4-1BB co-signaling domain that includes a sequence that is at least 90%, at least 95%, at least 98% identical to or identical to: KRGRKKLLYIFKQPFMRPVQTTQEEDGCSCRFPEEEEGGCEL (SEQ ID NO:3). The costimulatory domain(s) are located between the transmembrane domain and the CD3ζ signaling domain. The CD3ζ signaling domain can have the sequence:

(SEQ ID NO: 4)
RVKFSRSADAPAYQQGQNQLYNELNLGRREEYDVLDKRRGRDPEMGGKPRR
KNPQEGLYNELQKDKMAEAYSEIGMKGERRRGKGHDGLYQGLSTATKDTYD
ALHMQALPPR.

TABLE 1
CD3ζ Domain and Examples of Costimulatory
Domains

| Name | Accession | Length | Sequence |
|---|---|---|---|
| CD3ζ | J04132.1 | 112 aa | RVKFSRSADAPAYQQGQNQLY |
| | | | NELNLGRREEYDVLDKRRGRD |
| | | | PEMGGKPRRKNPQEGLYNELQ |
| | | | KDKMAEAYSEIGMKGERRRGK |
| | | | GHDGLYQGLSTATKDTYDALH |
| | | | MQALPPR (SEQ ID NO: 4) |
| CD28 | NM_006139 | 41 aa | RSKRSRLLHSDYMNMTPRRPG |
| | | | PTRKHYQPYAPPRDFAAYRS |
| | | | (SEQ ID NO: 1) |
| CD28gg* | NM_006139 | 41 aa | RSKRSRGGHSDYMNMTPRRPG |
| | | | PTRKHYQPYAPPRDFAAYRS |
| | | | (SEQ ID NO: 2) |
| 41BB | NM_001561 | 42 aa | KRGRKKLLYIFKQPFMRPVQT |
| | | | TQEEDGCSCRFPEEEEGGCEL |
| | | | (SEQ ID NO: 3) |
| OX40 | | 42 aa | ALYLLRRDQRLPPDAHKPPGG |
| | | | GSFRTPIQEEQADAHSTLAKI |
| | | | (SEQ ID NO: 5) |

In various embodiments: the costimulatory domain is selected from the group consisting of: a costimulatory domain depicted in Table 1 or a variant thereof having 1-5 (e.g., 1 or 2) amino acid modifications, a CD28 costimulatory domain or a variant thereof having 1-5 (e.g., 1 or 2) amino acid modifications, a 4-1BB costimulatory domain or a variant thereof having 1-5 (e.g., 1 or 2) amino acid modifications and an OX40 costimulatory domain or a variant thereof having 1-5 (e.g., 1 or 2) amino acid modifications. In certain embodiments, a 4-1BB costimulatory domain or a variant thereof having 1-5 (e.g., 1 or 2) amino acid modifications in present. In some embodiments there are two costimulatory domains, for example a CD28 co-stimulatory domain or a variant thereof having 1-5 (e.g., 1 or 2) amino acid modifications (e.g., substitutions) and a 4-1BB co-stimulatory domain or a variant thereof having 1-5 (e.g., 1 or 2) amino acid modifications (e.g., substitutions). In various embodiments the 1-5 (e.g., 1 or 2) amino acid modification are substitutions. The costimulatory domain is amino terminal to the CD3ζ signaling domain and in some cases a short linker consisting of 2-10, e.g., 3 amino acids (e.g., GGG) is positioned between the costimulatory domain and the CD3ζ signaling domain.

The CD3ζ Signaling domain can be any domain that is suitable for use with a CD3ζ signaling domain. In some cases, the CD3ζ signaling domain includes a sequence that is at least 90%, at least 95%, at least 98% identical to or identical to: RVKFSRSADAPAYQQGQNQLYNELNLGRREEYDVLDKRRGRDPEMGGKPRRKNPQEG LYNELQKDKMAEAYSEIGMKGERRRGKGHDGLYQGLSTATKDTYDALHMQALPPR (SEQ ID NO:4). In some cases, the CD3ζ signaling has 1, 2, 3, 4 of 5 amino acid changes (preferably conservative) compared to SEQ ID NO:4.

The present invention also provides a method comprising:

a. providing a population of human T cells harboring a nucleic acid molecule encoding a chimeric antigen receptor; and

b. culturing the population of human T cells for at least one day in a culture medium comprising one or more exogenously added cytokines and a compound that inhibits the activity of human PP2A, as described below and herein.

In some embodiments, the exogenously added cytokines are selected from the group consisting of IL-2, IL-15, IL-7 and IL-21. In some embodiments, exogenously added IL-2 is present at a concentration of less than 50 U/ml.

In some embodiments, the population of human T cells is cultured for at least 5 days in the culture medium. In some embodiments, exogenously added IL-15 is present at a concentration of at least 10 ng/ml. In some embodiments, the population of T cells comprises tumor infiltrating lymphocytes.

In some embodiments, the culture media comprises exogenously added IL-2 at a concentration of less than 10 U/ml. In some embodiments, the culture media comprises exogenously added IL-2 at a concentration of less than 1 U/ml. In some embodiments, the culture medium comprises exogenously added IL-7 at concentration of less than 5 ng/ml. In some embodiments, the culture medium comprises no exogenously added IL-7. In some embodiments, the culture medium comprises no exogenously added IL-21. In some embodiments, the culture medium comprises no exogenously added IL-2.

In some embodiments, the population of cells is cultured in the culture medium for at least five days and less than 40 days. In some embodiments, the population of cells is cultured in the culture medium for at least five days and less than 30 days. In some embodiments, the population of cells is cultured for a period of time sufficient to expand the population less than 100-fold.

In some embodiments, the step of providing a culture of T cells harboring a nucleic acid molecule encoding a chimeric antigen receptor comprises activating a population of T by exposing the population of T cells to antibodies targeted to human CD3 and antibodies targeted to human CD28 and subsequently or simultaneously exposing the population of T cells to a vector comprising a nucleic acid molecule encoding a chimeric antigen receptor. In some embodiments, the CD3 and CD28 antibodies are present on a solid support. In some embodiments, the vector is a lentiviral vector.

In some embodiments, the provided population of human T cells are prepared by a method comprising obtaining a sample of PBMC from a human patient, treating the obtained PBMC to isolate a population of cells enriched for central memory T cells; memory stem T cells, and naïve T cells, and transducing at least a portion of the isolated population of cells to with a viral vector comprising an nucleic acid molecule encoding a chimeric antigen receptor. In some embodiments, the step of treating the sample of PBMC to isolate a population of cells enriched for central memory T cells; memory stem T cells, and naïve T cells comprises: depleting the sample of PBMC of cells expressing CD14 and cells expressing CD25 and enriching for cells expressing CD62L to create a population of cells comprising: central memory T cells; memory stem T cells, and naïve T cells. In some embodiments, treating the sample of PBMC to isolate a population of cells enriched for central memory T cells; memory stem T cells, and naïve T cells does not comprise depleting cells expressing CD45RA.

In some embodiments, the population of human T cells are autologous to the patient. In some embodiments, the population of human T cells are allogenic to the patient.

In some embodiments, the chimeric antigen receptor is targeted to CD19.

The present invention also provides a method of enhancing the function of CART cells in a subject afflicted with cancer comprising administering to the subject a PP2A inhibitor so as to thereby enhance the function of the CAR T cells. In some such embodiments, and in embodiments described below and herein, a CAR T cell therapy may be administered concurrently with, prior to, or after administration of a PP2A inhibitor. In some such embodiments, and in embodiments described below and herein, a CAR T cell therapy may be administered concurrently with, prior to, or after administration of an additional anti-cancer therapy.

The present invention also provides a method of treating a subject afflicted with cancer comprising administering to the subject an effective amount of a PP2A inhibitor so as to thereby treat the cancer, wherein the cancer is susceptible to CAR T cell therapy, and wherein a CAR T cell therapy is administered to the subject concurrently with, prior to, or after administration of a PP2A inhibitor.

The present invention also provides a method of treating a subject afflicted with cancer and receiving anti-cancer therapy comprising administering to the subject an effective amount of PP2A inhibitor effective to enhance treatment relative to the anti-cancer therapy alone, wherein the cancer is susceptible to CAR T cell therapy, and wherein a CAR T cell therapy is administered to the subject concurrently with, prior to, or after administration of a PP2A inhibitor. In some such embodiments, the anti-cancer therapy is an anti-PD-1 agent. For instance, in some such embodiments, the anti-PD-1 agent is an anti-PD-1 monoclonal antibody, e.g., Nivolumab.

In some embodiments, the anti-cancer therapy can be an antibody or antibody fragment that binds to PD1, PD-L1, PD-L2 or CTLA4 (e.g., ipilimumab (also referred to as MDX-010 and MDX-101, and marketed as Yervoy®; Bristol-Myers Squibb; Tremelimumab (IgG2 monoclonal antibody available from Pfizer, formerly known as ticilimumab, CP-675,206).).

In some embodiments, the anti-cancer therapy is an inhibitor of PD1, e.g., an inhibitor of the interaction of PD1 and one of its natural ligands.

PD1 is an inhibitory member of the CD28 family of receptors that also includes CD28, CTLA-4, ICOS, and BTLA. PD1 is expressed on activated B cells, T cells and myeloid cells (Agata et al. 1996 Int. Immunol 8:765-75). Two ligands for PD1, PD-L1 and PD-L2 have been shown to downregulate T cell activation upon binding to PD1 (Freeman et a. 2000 J Exp Med 192:1027-34; Latchman et al. 2001 Nat Immunol 2:261-8; Carter et al. 2002 Eur J Immunol 32:634-43). Exemplary ligands include nivolumab (also referred to as BMS-936558 or MDX1106; Bristol-Myers Squibb), a fully human IgG4 monoclonal antibody which specifically blocks PD1. Nivolumab (clone 5C4) and other human monoclonal antibodies that specifically bind to PD1 are disclosed in U.S. Pat. No. 8,008,449 and WO2006/121168. Pidilizumab (CT-011; Cure Tech) is a humanized IgGlk monoclonal antibody that binds to PD1Pidilizumab and other humanized anti-PD1 monoclonal antibodies are disclosed in WO2009/101611. Lambrolizumab (also referred to as MK03475; Merck) is a humanized IgG4 monoclonal antibody that binds to PD1. Lambrolizumab and other humanized anti-PD1 antibodies are disclosed in U.S. Pat. No. 8,354,509 and WO2009/114335. MDPL3280A (Genentech/Roche) is a human Fc optimized IgG1 monoclonal antibody that binds to PD-L1. MDPL3280A and other human monoclonal antibodies to PD-L1 are disclosed in U.S. Pat. No. 7,943,743 and U.S Publication No.: 20120039906. Other anti-PD-L1 binding agents include YW243.55.570 (heavy and light chain variable regions are shown in SEQ ID NOs 20 and 21 in WO2010/077634) and MDX-1 105 (also referred to as BMS-936559, and, e.g., anti-PD-L1 binding agents disclosed in WO2007/005874). AMP-224 (B7-DCIg; Amplimmune; e.g., disclosed in WO2010/027827 and WO2011/066342), is a PD-L2 Fc fusion soluble receptor that blocks the interaction between PD1 and B7-H1. Other anti-PD1 antibodies include AMP 514 (Amplimmune), among others, e.g., anti-PD1 antibodies disclosed in U.S. Pat. No. 8,609,089, US 2010028330, and/or US 20120114649.

In some embodiments, the anti-PD-1 agent is a monoclonal antibody selected from pembrolizumab, nivolumab, avelumab, durvalumab, andazetolizumab. In some embodiements, the anti-PD-1 agent is BMS-1001 or BMS 1166.

In some embodiments, an anti-cancer agent for use in provided methods is any of those disclosed in US Pharm. 2018:(43(2)27-31, herein incorporated by reference in its entirety.

In some embodiments, compositions and methods provided herein are contemplated as providing effective treatments for diseases such as cancer (e.g., mantel cell lymphoma). Thus, the present invention provides a method of treating cancer in a subject in need thereof, the method including administering to a subject a therapeutically effective amount of a recombinant protein as provided herein, including embodiments thereof, thereby treating cancer in the subject.

The recombinant proteins provided herein including embodiments thereof, may be administered in combination with additional therapeutic agents. Thus, in embodiments, the method provided herein, including embodiments thereof, further includes administering to the subject a second therapeutic agent.

In some embodiments, the cancer is a neuroblastoma, for instance a current/refractory neuroblastoma. In some embodiments, the cancer is a glioma. In some embodiments, a cancer is a gastric cancer. In some embodiments, the cancer is a kidney cancer. In some embodiments, the cancer is a metastatic renal cell carcinoma. In some embodiments, the cancer is an ovarian cancer. In some embodiments, the cancer is a sarcoma. In some embodiments, the cancer is a glioblastoma. In some embodiments, the cancer is an osteosarcoma. In some embodiments, the cancer is a metastatic colon cancer. In some embodiments, the cancer is a melanoma. In some embodiments, the cancer is a medulloblastoma.

In some embodiments, the cancer is a cancer of the brain, for instance a glioblastoma. In some embodiments, the cancer is a cancer of the pancreas. In some embodiments, the cancer is a pancreatic ductal adenocarcinoma. In some embodiments, the cancer is a cancer of the breast. In some embodiments, the cancer is a HER-2 positive cancer. In some such embodiments, the HER-2 positive cancer is a HER-2 positive sarcoma. In some embodiments, the cancer is an MSLN-positive cancer. In some embodiments, the cancer is a CD133-positive malignancy. In some embodiments, the cancer is a pleural mesothelioma, for instance a malignant pleural mesothelioma. In some embodiments, the cancer is a cancer of the liver. In some embodiments, the cancer is a liver metastatis. In some embodiments, the cancer is a gastric cancer. In some embodiments, the cancer is hepatocellular carcinoma, human osteosarcoma, primary liver cancer, gastric cancer, ovarian cancer, endometrial cancer, colorectal cancer, non-small cell lung cancer, soft-tissue sarcoma, seminoma, breast cancer, lymphoma, fibrosarcoma, neuroblastoma, mucinous ovarian cancer, urothelial bladder cancer, squamous cell carcinoma of the uterine cervix, diffuse large cell lymphoma, lung adenoma, hepatoma, intestinal cancer, fibrosarcoma, prostate cancer, angiomyolipoma, mammary adenocarcinoma or acute myelogenous leukemia.

In some embodiments, the cancer is hepatocellular carcinoma, human osteosarcoma, primary liver cancer, gastric cancer, ovarian cancer, endometrial cancer, colorectal cancer, non-small cell lung cancer, soft-tissue sarcoma, seminoma, breast cancer, lymphoma, fibrosarcoma, or neuroblastoma.

In some embodiments, the cancer is mucinous ovarian cancer, urothelial bladder cancer, squamous cell carcinoma of the uterine cervix, or diffuse large cell lymphoma.

In some embodiments, the cancer is lung adenoma, hepatoma, hepatocellular carcinoma, intestinal cancer, lymphoma, fibrosarcoma, prostate cancer, angiomyolipoma, or mammary adenocarcinoma.

In some embodiments, the cancer is acute myelogenous leukemia.

In some embodiments, the cancer is breast cancer, colon cancer, large cell lung cancer, adenocarcinoma of the lung, small cell lung cancer, stomach cancer, liver cancer, ovary adenocarcinoma, pancreas carcinoma, prostate carcinoma, promylocytic leukemia, chronic myelocytic leukemia, acute lymphocytic leukemia, colorectal cancer, ovarian cancer, lymphoma, non-Hodgkin's lymphoma or Hodgkin's lymphoma.

In some embodiments, a cancer is any of those disclosed in Mirzaei et al. 2017 Chimeric Antigen Receptors T Cell Therapy in Solid Tumor: Challenges and Clinical Applications, Frontiers in Immunobiology, vol. B, Article 1850, p. 1-13, the entirely of which is incorporated herein by reference.

In some embodiments of any of the above methods, the cancer is a cancer of the blood.

In some embodiments, the cancer of the blood is a lymphoma.

In some embodiments, the cancer of the blood is a leukemia. In some embodiments, the leukemia is acute. In some embodiments, the leukemia is chronic. In some embodiments the leukemia is selected from acute myeloid leukemia (AML), acute lymphoid leukemia (ALL), chronic myeloid leukemia (CIVIL), or chronic lymphoid leukemia (CLL).

In some embodiments, the leukemia is an acute myeloid leukemia (AML).

In some embodiments, the leukemia is an acute lymphoid leukemia (ALL).

In some embodiments, the leukemia is a chronic myeloid leukemia (CML).

In some embodiments, the leukemia is a chronic lymphoid leukemia (CLL).

In some embodiments, the leukemia is promylocytic leukemia.

In some embodiments, the cancer is lymphoma, leukemia, or myeloma. In some embodiments, the cancer is lymphoma. In some embodiments, the cancer is leukemia. In some embodiments, the cancer is myeloma.

In some embodiments, the lymphoma is non-Hodgkin's lymphoma. In some embodiments, the non-Hodgkin's lymphoma is mantle cell lymphoma, follicular lymphoma, diffuse large B-cell lymphoma, marginal zone lymphoma or Burkitt's lymphoma. In some embodiments, the non-Hodgkin's lymphoma is mantle cell lymphoma. In some embodiments, the non-Hodgkin's lymphoma is follicular lymphoma. In some embodiments, the non-Hodgkin's lymphoma is diffuse large B-cell lymphoma. In some embodiments, the non-Hodgkin's lymphoma is marginal zone lymphoma. In embodiments, the non-Hodgkin's lymphoma is Burkitt's lymphoma.

In some embodiments, the leukemia is lymphoblastic leukemia, chronic lymphocytic leukemia or hairy cell leukemia. In some embodiments, the leukemia is lymphoblastic leukemia. In some embodiments, the leukemia is chronic lymphocytic leukemia. In some embodiments, the leukemia is hairy cell leukemia.

In some embodiments, the above methods further comprise administering an anti-cancer therapy concurrently with, prior to, or after the PP2A inhibitor. As described above and herein, in some such embodiments, the anti-cancer therapy comprises administration of a checkpoint inhibitor. In some embodiments, the checkpoint inhibitor is an anti-PD-1 agent such as any of those disclosed above and herein. In some embodiments, the anti-PD-1 agent is an anti-PD-1 monoclonal antibody, e.g., Nivolumab.

In some embodiments, the present invention provides a method of treating a subject afflicted with cancer comprising administering to the subject an effective amount of an anti-PD-1 agent (e.g., nivolumab) and an effective amount of a PP2A inhibitor, wherein the amounts when taken together are effective to enhance CAR T cell function in a subject that is undergoing, has undergone, and/or will undergo, a CAR T cell therapy.

In some embodiments of the above methods, the amount of PP2A inhibitor and the amount of anti-PD-1 agent are each periodically administered to the subject.

In some embodiments of the above methods, the amount of PP2A inhibitor and the amount of anti-PD-1 agent are administered simultaneously, separately or sequentially.

In some embodiments of the above methods, the amount of anti-PD-1 agent and the amount of PP2A inhibitor when administered together is more effective to treat the subject than when either agent in the same amount is administered alone.

In some embodiments of the above methods, the PP2A inhibitor enhances the chemotherapeutic effect of the anti-PD-1 agent.

In some embodiments of the above methods, the anti-PD-1 agent is an anti-PD-1 monoclonal antibody. For instance, in some embodiments, the anti-PD-1 monoclonal antibody is nivolumab.

In some embodiments of the above methods, the PP2A inhibitor is of the following structure:

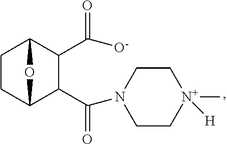 [SMILES: C[NH+]1CCN(C(=O)C2C(C(=O)[O-])[C@H]3CC[C@@H]2O3)CC1]

or a pharmaceutically acceptable salt thereof

In some embodiments of any of the above methods, the PP2A inhibitor is of the following structure:

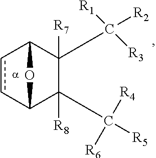

wherein
bond α is present or absent;
R_{1 and} R₂ together are ═O;
R₃ is OH, O⁻, OR₉, O(CH₂)₁₋₆R₉, SH, S⁻, or SR₉,
   wherein R₉ is H, alkyl, alkenyl, alkynyl or aryl;
R₄ is

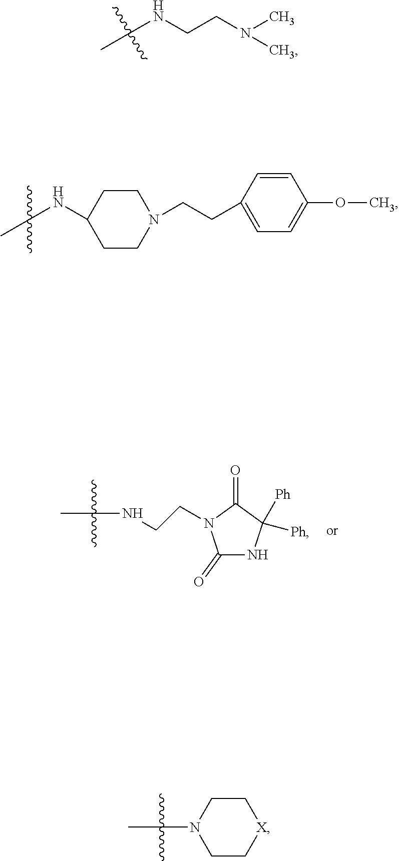

where X is O, S, NR₁₀, N⁺HR₁₀ or N₁₀R₁₀,
   where each R₁₀ is independently H, alkyl, alkenyl, alkynyl, aryl,

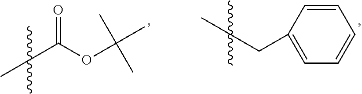

—CH₂CN, —CH₂CO₂R₁₁, or —CH₂COR₁₁, wherein each R₁₁ is independently H, alkyl, alkenyl or alkynyl;
R₅ and R₆ taken together are ═O;
R₇ and R₈ are each H,
   or a salt, zwitterion, or ester thereof.

In some embodiments, the compound has the structure:

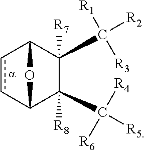

In some embodiments, bond α in the compound is present.

In some embodiments, bond α in the compound is absent.

In some embodiments, R₃ is OH, O⁻, or OR₉, wherein R₉ is alkyl, alkenyl, alkynyl or aryl;
R₄ is

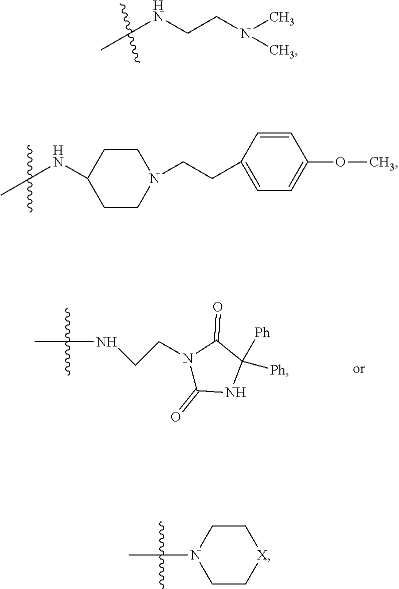

wherein X is O, S, NR₁₀, N⁺HR₁₀ or N⁺R₁₀R₁₀,
wherein each R₁₀ is independently H, alkyl, alkenyl, alkynyl, aryl,

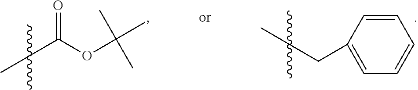

In some embodiments, R₃ is OH, O— or OR₉, wherein R₉ is H, methyl, ethyl or phenyl.

In some embodiments, R₃ is OH, O⁻ or OR₉, wherein R₉ is methyl.

In some embodiments, R₄ is

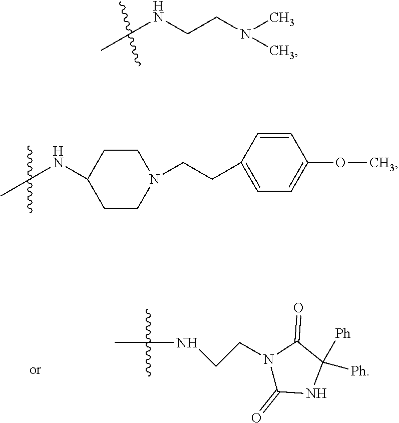

In some embodiments, R₄ is

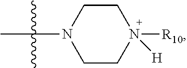

wherein R₁₀ is H, alkyl, alkenyl, alkynyl, aryl, or

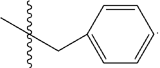 [SMILES: *Cc1ccccc1]

In some embodiments, R₄ is

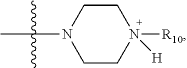

wherein R₁₀ is —H, —CH₃, —CH₂CH₃, or

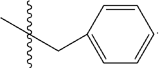 [SMILES: *Cc1ccccc1]

In some embodiments, R₄ is

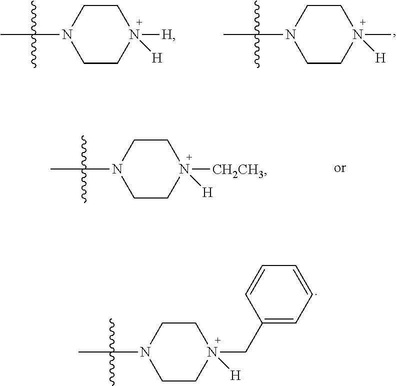

In some embodiments, R₄ is

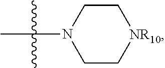

wherein R₁₀ is H, alkyl, alkenyl, alkynyl, aryl,

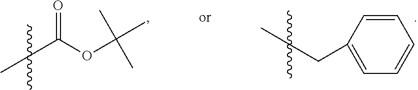

In some embodiments, R₄ is

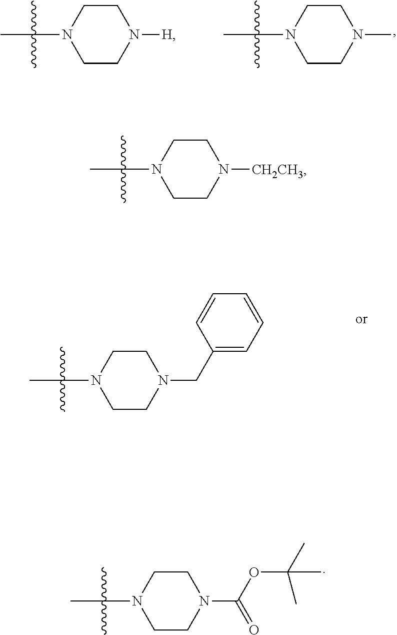

In some embodiments, R₄ is

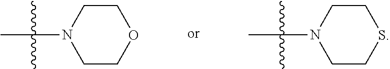 [SMILES: *N1CCOCC1 ; "or" ; *N1CCSCC1]

In some embodiments, the compound has the structure:

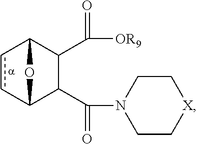

wherein
bond α is present or absent;
R₉ is present or absent and when present is H, alkyl, alkenyl, alkynyl or phenyl; and
X is O, NR₁₀, NH⁺R₁₀ or N⁺R₁₀R₁₀,
   wherein each R₁₀ is independently H, alkyl, substituted alkyl, alkenyl, substituted alkenyl, alkynyl, substituted alkynyl, aryl,

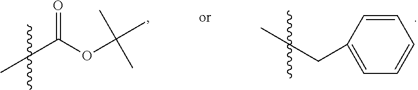

—CH₂CN, —CH₂CO₂R₁₂, or —CH₂COR₁₂,
wherein R₁₂ is H or alkyl,
or a salt, zwitterion or ester thereof.

In some embodiments, the compound has the structure:

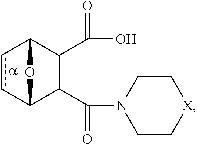

wherein
bond α is present or absent;
X is O or NR₁₀,
   where each R₁₀ is independently H, alkyl, substituted alkyl, alkenyl, substituted alkenyl, alkynyl, substituted alkynyl, aryl,

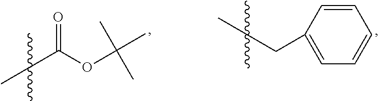

—CH₂CN, —CH₂CO₂R₁₂, or —CH₂COR₁₂,
where R₁₂ is H or alkyl,
   or a salt, zwitterion or ester thereof.

In some embodiments, the compound has the structure:

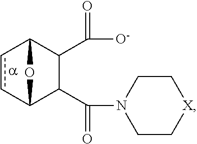

wherein
bond α is present or absent;
X is O or NH⁺R₁₀,
   where R₁₀ is H, alkyl, substituted alkyl, alkenyl, substituted alkenyl, alkynyl, substituted alkynyl, aryl,

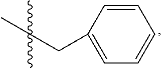 [SMILES: *Cc1ccccc1]

—CH₂CN, —CH₂CO₂R₁₂, or —CH₂COR₁₂,
where R₁₂ is H or alkyl,
   or a salt, zwitterion or ester thereof.

In some embodiments, the compound has the structure

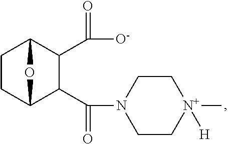 [SMILES: C[NH+]1CCN(C(=O)C2C(C(=O)[O-])[C@H]3CC[C@@H]2O3)CC1]

or a salt or ester thereof.

In some embodiments, the compound has the structure

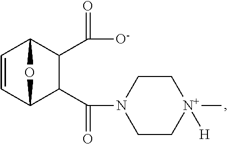 [SMILES: C[NH+]1CCN(C(=O)C2C(C(=O)[O-])[C@H]3C=C[C@@H]2O3)CC1]

or a salt or ester thereof.

Cancers susceptible to CAR T cell therapy include, but are not limited to, cancers which have been shown to be amenable to CAR T cell therapy in pre-clinical or clinical trials.

In some embodiments, the amount of PP2A inhibitor is effective to reduce a clinical symptom of the cancer in the subject.

In some embodiments, treating comprises increasing the percentage of CAR+/CD8+/INFγ+ in the subject.

The analogs of LB-100 disclosed herein have analogous activity to LB-100 and work similarly in the methods described herein.

In some embodiments, the method wherein the subject is administered a pharmaceutical composition comprising a compound of the present invention and at least one pharmaceutically acceptable carrier for treating the cancer in the subject.

In some embodiments, the pharmaceutical composition wherein the pharmaceutically acceptable carrier comprises a liposome.

In some embodiments, the pharmaceutical composition wherein the compound is contained in a liposome or microsphere.

In some embodiments is provided a pharmaceutical composition including a therapeutically effective amount of a recombinant protein as described herein, including embodiments thereof, and a pharmaceutically acceptable excipient.

In some embodiments, an isolated nucleic acid encoding a recombinant protein as described herein, including embodiments thereof is provided.

In some embodiments, the pharmaceutical composition comprises the PP2A inhibitor and an anti-PD-1 agent, for instance an anti-PD-1 monoclonal antibody such as nivolumab.

In some embodiments of any of the above methods or uses, the subject is a human.

In some embodiments of any of the above methods or uses, the compound and/or the anti-PD-1 agent is orally administered to the subject.

The present invention provides a PP2A inhibitor for use in enhancing the function of CART cells.

The present invention provides a PP2A inhibitor for use in enhancing the function of CAR T cells in a subject afflicted with cancer.

The present invention provides a PP2A inhibitor for use in treating a subject afflicted with cancer, wherein the cancer is susceptible to CAR T cell therapy.

The present invention provides a PP2A inhibitor in combination with an anti-PD-1 agent for use in treating a subject afflicted with cancer.

The present invention also provides a method of optimizing the concentration of LB100 in the bloodstream of a subject who has been administered a dosage of LB100 comprising:
(a) measuring the plasma concentration of LB100 in the subject;
(b) determining whether a further LB100 dose needs to be administered to the subject based on whether the measurement in (a); and
(c) administering a further dosage or dosages of the LB100 as necessary based on the determination in (b).

In some embodiments, the above step (b) comprises determining whether a further LB100 dose needs to be administered to the subject based on whether the measurement in (a) is above, below or equal to the Minimum Effective Concentration (MEC) of LB100.

In some embodiments, the initial dose of LB100 administered to the subject is an amount of from 0.1 mg/m² to 5 mg/m².

In some embodiments, the further dose of LB100 administered to the subject is an amount of from 0.1 mg/m² to 5 mg/m².

In some embodiments, the compound is administered at a dose of 0.25 mg/m², 0.5 mg/m², 0.83 mg/m², 1.25 mg/m², 1.75 mg/m², 2.33 mg/m², of 3.1 mg/m².

In some embodiments, the compound is administered at a dose of 2.33 mg/m².

In some embodiments, the compound is administered for 3 days every 3 weeks.

In some embodiments, the further dose of LB100 administered to the subject is an amount 25% less than the initial dose.

In some embodiments, the further dose of LB100 administered to the subject is an amount 50% less than the initial dose.

In some embodiments, the further dose of LB100 administered to the subject is an amount 75% less than the initial dose.

In some embodiments, the further dose of LB100 administered to the subject is an amount 25% more than the initial dose.

In some embodiments, the further dose of LB100 administered to the subject is an amount 50% more than the initial dose.

In some embodiments, the further dose of LB100 administered to the subject is an amount 75% more than the initial dose.

In some embodiments, the subject is further treated with an anti-cancer therapy concurrently with, prior to, or after the administering.

Examples of anti-cancer therapy include radiation therapy or chemotherapy, targeted therapy to promote antigen relase, vaccination to promote antigen presentation, agonist for co-stimulatory molecules or blockade of co-inhibitory molecules to amplify T cell activation, trafficking inhibition of regulatory T cells or myeloid derived suppressor cells, anti-vascular endothelial growth factor to stimulate intratumoral T cell infiltration, adoptive cell transfer to increase cancer recognition by T cell infiltration, or stimulate tumor killing. Further examples may be found in Swart et al., 2016; Topalian et al., 2015; and Tsiatas et al. 2016.

In some embodiments, the anti-cancer therapy comprises immunotherapy. The term “immunotherapy” refers to the treatment of a subject afflicted with a disease by a method comprising inducing, enhancing, suppressing or otherwise modifying an immune response. Immunotherapy agents may include antibody agents targeting one or more of CTLA-4, PD-1, PD-L1, GITR, 0C40, LAG-3, KIR, TIM-3, B7-H3, B7-H4, CD28, CD40; and CD137.

In some embodiments, the anti-cancer therapy comprises administering an anti-cancer agent.

In some embodiments, the anti-cancer agent is an immune checkpoint modulator. The term “immune checkpoint modulator” refers to an agent that interacts directly or indirectly with an immune checkpoint. Immune checkpoint modulators may be administered to overcome inhibitory signals and permit and/or augment an immune attach against cancer cells. In some embodiments, an immune checkpoint modulator increases an immune effector response (e.g. cytotoxic T cell response). In some embodiments, an immune checkpoint modulator reduces, removes, or prevents immune tolerance to one or more antigens. For example, immune checkpoint modulators may facilitate immune cell responses by decreasing, inhibiting, or abrogating signaling by negative immune response regulators by stimulating or enhancing signaling of positive regulators of immune response, or by preventing autoimmune responses and limiting immune cell-mediated tissue damage.

In some embodiments, the anti-cancer agent comprises an antibody or an antigen-binding portion thereof.

In some embodiments, the antibody or antigen-binding portion thereof binds specifically to a Programmed Death-a (PD-1) receptor and inhibits PD-1 activity (“anti-PD-1 antibody”). In some embodiments, the anti-PD-1 antibody is nivolumab or pembrolizumab.

The present invention also provides a method of treating a tumor or cancer in a subject comprising administering to the subject an effective amount of a PP2A inhibitor, wherein the tumor or cancer is susceptible to treatment by an immune response.

In some embodiments, the PP2A inhibitor has the structure:

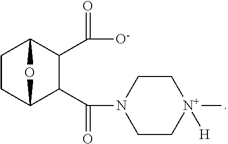 [SMILES: C[NH+]1CCN(C(=O)C2C(C(=O)[O-])[C@H]3CC[C@@H]2O3)CC1]

Analogs of LB-100 have analogous activity to LB-100 and exhibit similar effects in the methods described herein. Such analogs include the compounds described in PCT International Application Publication No. WO 2008/097561, published Aug. 14, 2008; PCT International Application Publication No. WO 2010/014254, published Feb. 4, 2010; PCT International Application Publication No. WO 2015/073802, published May 21, 2015; and PCT International Application Publication No. WO 2016/186963, published Nov. 24, 2016, the contents of each of which are hereby incorporated by reference.

Compounds which act as prodrugs for the in vivo delivery of LB-100 and/or endothal have analogous activity to LB-100 and exhibit similar effects in the methods described herein. More specifically, administration of the prodrug provides a similar effect to the administration of LB-100. Pro-drugs of LB-100 and/or endothal include the compounds described in PCT International Application Publication No. WO 2015/073802, published May 21, 2015; and PCT International Application Publication No. WO 2016/186963, published Nov. 24, 2016, the contents of each of which are hereby incorporated by reference.

In some embodiments, the method further comprising administering one or more additional anti-cancer agent.

The present invention also provides a method of treating a subject afflicted with cancer comprising administering to the subject an effective amount of a PP2A inhibitor in combination with an effective amount of an anti-cancer therapy, wherein the amounts when taken together are effective to treat the subject.

The present invention also provides a method of treating a subject afflicted with cancer and receiving anti-cancer therapy comprising administering to the subject an effective amount of PP2A inhibitor effective to enhance treatment relative to the anti-cancer therapy alone.

In some embodiments, the cancer is susceptible to treatment by an immune response.

The compounds used in the method of the present invention are protein phosphatase 2A (PP2A) inhibitors. Methods of preparation may be found in Lu et al., 2009; U.S. Pat. Nos. 7,998,957 B2; and 8,426,444 B2. Compound LB-100 is an inhibitor of PP2A in vitro in human cancer cells and in xenografts of human tumor cells in mice when given parenterally in mice. LB-100 inhibits the growth of cancer cells in mouse model systems.

As used herein, a “symptom” associated cancer includes any clinical or laboratory manifestation associated with cancer and is not limited to what the subject can feel or observe.

As used herein, “treatment of the diseases” or “treating” encompasses inducing prevention, inhibition, regression, or stasis of the disease or a symptom or condition associated with the disease.

As used herein, “inhibition” of disease progression or disease complication in a subject means preventing or reducing the disease progression and/or disease complication in the subject.

As used herein, “alkyl” is intended to include both branched and straight-chain saturated aliphatic hydrocarbon groups having the specified number of carbon atoms. Thus, C₁-Cₙ as in “C₁-Cₙ alkyl” is defined to include groups having 1, 2 , . . . , n-1 or n carbons in a linear or branched arrangement, and specifically includes methyl, ethyl, propyl, butyl, pentyl, hexyl, heptyl, isopropyl, isobutyl, sec-butyl and so on. An embodiment can be C₁-C₂₀ alkyl, C₂-C₂₀ alkyl, C₃-C₂₀ alkyl, C₄-C₂₀ alkyl and so on. An embodiment can be C₁-C₃₀ alkyl, C₂-C₃₀ alkyl, C₃-C₃₀ alkyl, C₄-C₃₀ alkyl and so on. “Alkoxy” represents an alkyl group as described above attached through an oxygen bridge.

The term “alkenyl” refers to a non-aromatic hydrocarbon radical, straight or branched, containing at least 1 carbon to carbon double bond, and up to the maximum possible number of non-aromatic carbon-carbon double bonds may be present. Thus, C₂-Cₙ alkenyl is defined to include groups having 1, 2 . . . , n-1 or n carbons. For example, “C₂-C₆ alkenyl” means an alkenyl radical having 2, 3, 4, 5, or 6 carbon atoms, and at least 1 carbon-carbon double bond, and up to, for example, 3 carbon-carbon double bonds in the case of a C₆ alkenyl, respectively. Alkenyl groups include ethenyl, propenyl, butenyl and cyclohexenyl. As described above with respect to alkyl, the straight, branched or cyclic portion of the alkenyl group may contain double bonds and may be substituted if a substituted alkenyl group is indicated. An embodiment can be C₂-C₁₂ alkenyl, C₃-C₁₂ alkenyl, C₂-C₂₀ alkenyl, C₃-C₂₀ alkenyl, C₂-C₃₀ alkenyl, or C₃-C₃₀ alkenyl.

The term “alkynyl” refers to a hydrocarbon radical straight or branched, containing at least 1 carbon to carbon triple bond, and up to the maximum possible number of non-aromatic carbon-carbon triple bonds may be present. Thus, C₂-Cₙ alkynyl is defined to include groups having 1, 2 . . . , n-1 or n carbons. For example, “C₂-C₆ alkynyl” means an alkynyl radical having 2 or 3 carbon atoms, and 1 carbon-carbon triple bond, or having 4 or 5 carbon atoms, and up to 2 carbon-carbon triple bonds, or having 6 carbon atoms, and up to 3 carbon-carbon triple bonds. Alkynyl groups include ethynyl, propynyl and butynyl. As described above with respect to alkyl, the straight or branched portion of the alkynyl group may contain triple bonds and may be substituted if a substituted alkynyl group is indicated. An embodiment can be a C₂-Cₙalkynyl. An embodiment can be C₂-C₁₂ alkynyl or C₃-C₁₂ alkynyl, C₂-C₂₀ alkynyl, C₃-C₂₀ alkynyl, C₂-C₃₀ alkynyl, or C₃-C₃₀ alkynyl.

As used herein, “aryl” is intended to mean any stable monocyclic or bicyclic carbon ring of up to 10 atoms in each ring, wherein at least one ring is aromatic. Examples of such aryl elements include phenyl, naphthyl, tetrahydro-naphthyl, indanyl, biphenyl, phenanthryl, anthryl or acenaphthyl. In cases where the aryl substituent is bicyclic and one ring is non-aromatic, it is understood that attachment is via the aromatic ring. The substituted aryls included in this invention include substitution at any suitable position with amines, substituted amines, alkylamines, hydroxys and alkylhydroxys, wherein the “alkyl” portion of the alkylamines and alkylhydroxys is a C₂-Cₙ alkyl as defined hereinabove. The substituted amines may be substituted with alkyl, alkenyl, alkynl, or aryl groups as hereinabove defined.

Each occurrence of alkyl, alkenyl, or alkynyl is branched or unbranched, unsubstituted or substituted.

The alkyl, alkenyl, alkynyl, and aryl substituents may be unsubstituted or unsubstituted, unless specifically defined otherwise. For example, a (C₁-C₆) alkyl may be substituted with one or more substituents selected from OH, oxo, halogen, alkoxy, dialkylamino, or heterocyclyl, such as morpholinyl, piperidinyl, and so on.

In the compounds of the present invention, alkyl, alkenyl, and alkynyl groups can be further substituted by replacing one or more hydrogen atoms by non-hydrogen groups described herein to the extent possible. These include, but are not limited to, halo, hydroxy, mercapto, amino, carboxy, cyano and carbamoyl.

The term “substituted” as used herein means that a given structure has a substituent which can be an alkyl, alkenyl, or aryl group as defined above. The term shall be deemed to include multiple degrees of substitution by a named substitutent. Where multiple substituent moieties are disclosed or claimed, the substituted compound can be independently substituted by one or more of the disclosed or claimed substituent moieties, singly or plurally. By independently substituted, it is meant that the (two or more) substituents can be the same or different.

It is understood that substituents and substitution patterns on the compounds of the instant invention can be selected by one of ordinary skill in the art to provide compounds that are chemically stable and that can be readily synthesized by techniques known in the art, as well as those methods set forth below, from readily available starting materials. If a substituent is itself substituted with more than one group, it is understood that these multiple groups may be on the same carbon or on different carbons, so long as a stable structure results.

As used herein, “administering” an agent may be performed using any of the various methods or delivery systems well known to those skilled in the art. The administering can be performed, for example, orally, parenterally, intraperitoneally, intravenously, intraarterially, transdermally, sublingually, intramuscularly, rectally, transbuccally, intranasally, liposomally, via inhalation, vaginally, intraoccularly, via local delivery, subcutaneously, intraadiposally, intraarticularly, intrathecally, into a cerebral ventricle, intraventicularly, intratumorally, into cerebral parenchyma or intraparenchchymally.

The following delivery systems, which employ a number of routinely used pharmaceutical carriers, may be used but are only representative of the many possible systems envisioned for administering compositions in accordance with the invention.

Injectable drug delivery systems include solutions, suspensions, gels, microspheres and polymeric injectables, and can comprise excipients such as solubility-altering agents (e.g., ethanol, propylene glycol and sucrose) and polymers (e.g., polycaprylactones and PLGA's).

Other injectable drug delivery systems include solutions, suspensions, gels. Oral delivery systems include tablets and capsules. These can contain excipients such as binders (e.g., hydroxypropylmethylcellulose, polyvinyl pyrilodone, other cellulosic materials and starch), diluents (e.g., lactose and other sugars, starch, dicalcium phosphate and cellulosic materials), disintegrating agents (e.g., starch polymers and cellulosic materials) and lubricating agents (e.g., stearates and talc).

Implantable systems include rods and discs, and can contain excipients such as PLGA and polycaprylactone.

Oral delivery systems include tablets and capsules. These can contain excipients such as binders (e.g., hydroxypropylmethylcellulose, polyvinyl pyrilodone, other cellulosic materials and starch), diluents (e.g., lactose and other sugars, starch, dicalcium phosphate and cellulosic materials), disintegrating agents (e.g., starch polymers and cellulosic materials) and lubricating agents (e.g., stearates and talc).

Transmucosal delivery systems include patches, tablets, suppositories, pessaries, gels and creams, and can contain excipients such as solubilizers and enhancers (e.g., propylene glycol, bile salts and amino acids), and other vehicles (e.g., polyethylene glycol, fatty acid esters and derivatives, and hydrophilic polymers such as hydroxypropylmethylcellulose and hyaluronic acid).

Dermal delivery systems include, for example, aqueous and nonaqueous gels, creams, multiple emulsions, microemulsions, liposomes, ointments, aqueous and nonaqueous solutions, lotions, aerosols, hydrocarbon bases and powders, and can contain excipients such as solubilizers, permeation enhancers (e.g., fatty acids, fatty acid esters, fatty alcohols and amino acids), and hydrophilic polymers (e.g., polycarbophil and polyvinylpyrolidone). In one embodiment, the pharmaceutically acceptable carrier is a liposome or a transdermal enhancer.

Solutions, suspensions and powders for reconstitutable delivery systems include vehicles such as suspending agents (e.g., gums, zanthans, cellulosics and sugars), humectants (e.g., sorbitol), solubilizers (e.g., ethanol, water, PEG and propylene glycol), surfactants (e.g., sodium lauryl sulfate, Spans, Tweens, and cetyl pyridine), preservatives and antioxidants (e.g., parabens, vitamins E and C, and ascorbic acid), anti-caking agents, coating agents, and chelating agents (e.g., EDTA).

As used herein, “pharmaceutically acceptable carrier” refers to a carrier or excipient that is suitable for use with humans and/or animals without undue adverse side effects (such as toxicity, irritation, and allergic response) commensurate with a reasonable benefit/risk ratio. It can be a pharmaceutically acceptable solvent, suspending agent or vehicle, for delivering the instant compounds to the subject.

The compounds used in the method of the present invention may be in a salt form. As used herein, a “salt” is a salt of the instant compounds which has been modified by making acid or base salts of the compounds. In the case of compounds used to treat an infection or disease, the salt is pharmaceutically acceptable. Examples of pharmaceutically acceptable salts include, but are not limited to, mineral or organic acid salts of basic residues such as amines; alkali or organic salts of acidic residues such as phenols. The salts can be made using an organic or inorganic acid. Such acid salts are chlorides, bromides, sulfates, nitrates, phosphates, sulfonates, formates, tartrates, maleates, malates, citrates, benzoates, salicylates, ascorbates, and the like. Phenolate salts are the alkaline earth metal salts, sodium, potassium or lithium. The term “pharmaceutically acceptable salt” in this respect, refers to the relatively non-toxic, inorganic and organic acid or base addition salts of compounds of the present invention. These salts can be prepared in situ during the final isolation and purification of the compounds of the invention, or by separately reacting a purified compound of the invention in its free base or free acid form with a suitable organic or inorganic acid or base, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. (See, e.g., Berge et al. (1977) “Pharmaceutical Salts”, J. Pharm. Sci. 66:1-19).

The present invention includes esters or pharmaceutically acceptable esters of the compounds of the present method. The term “ester” includes, but is not limited to, a compound containing the R—CO—OR′ group. The “R—CO—O” portion may be derived from the parent compound of the present invention. The “R” portion includes, but is not limited to, alkyl, alkenyl, alkynyl, heteroalkyl, aryl, and carboxy alkyl groups.

The present invention includes pharmaceutically acceptable prodrug esters of the compounds of the present method. Pharmaceutically acceptable prodrug esters of the compounds of the present invention are ester derivatives which are convertible by solvolysis or under physiological conditions to the free carboxylic acids of the parent compound. An example of a pro-drug is an alkly ester which is cleaved in vivo to yield the compound of interest.

The compound, or salt, zwitterion, or ester thereof, is optionally provided in a pharmaceutically acceptable composition including the appropriate pharmaceutically acceptable carriers.

As used herein, an “amount” or “dose” of an agent measured in milligrams refers to the milligrams of agent present in a drug product, regardless of the form of the drug product.

The National Institutes of Health (NIH) provides a table of Equivalent Surface Area Dosage Conversion Factors below (Table A) which provides conversion factors that account for surface area to weight ratios between species.

TABLE A
Equivalent Surface Area Dosage Conversion Factors

| | | To | | | | |
|---|---|---|---|---|---|---|
| | | Mouse | Rat | Monkey | Dog | Man |
| | | 20 g | 150 g | 3 kg | 8 kg | 60 kg |
| From | Mouse | 1 | ½ | ¼ | ⅙ | 1/12 |
| | Rat | 2 | 1 | ½ | ¼ | 1/7 |
| | Monkey | 4 | 2 | 1 | ⅗ | ⅓ |
| | Dog | 6 | 4 | 1⅔ | 1 | ½ |
| | Man | 12 | 7 | 3 | 2 | 1 |

As used herein, the term “therapeutically effective amount” or “effective amount” refers to the quantity of a component that is sufficient to yield a desired therapeutic response without undue adverse side effects (such as toxicity, irritation, or allergic response) commensurate with a reasonable benefit/risk ratio when used in the manner of this invention. The specific effective amount will vary with such factors as the particular condition being treated, the physical condition of the patient, the type of mammal being treated, the duration of the treatment, the nature of concurrent therapy (if any), and the specific formulations employed and the structure of the compounds or its derivatives.

Where a range is given in the specification it is understood that the range includes all integers and 0.1 units within that range, and any sub-range thereof. For example, a range of 77 to 90% is a disclosure of 77, 78, 79, 80, and 81% etc.

As used herein, “about” with regard to a stated number encompasses a range of +one percent to −one percent of the stated value. By way of example, about 100 mg/kg therefore includes 99, 99.1, 99.2, 99.3, 99.4, 99.5, 99.6, 99.7, 99.8, 99.9, 100, 100.1, 100.2, 100.3, 100.4, 100.5, 100.6, 100.7, 100.8, 100.9 and 101 mg/kg. Accordingly, about 100 mg/kg includes, in an embodiment, 100 mg/kg.

It is understood that where a parameter range is provided, all integers within that range, and tenths thereof, are also provided by the invention. For example, “0.2-5 mg/kg/day” is a disclosure of 0.2 mg/kg/day, 0.3 mg/kg/day, 0.4 mg/kg/day, 0.5 mg/kg/day, 0.6 mg/kg/day etc. up to 5.0 mg/kg/day.

For the foregoing embodiments, each embodiment disclosed herein is contemplated as being applicable to each of the other disclosed embodiments. Thus, all combinations of the various elements described herein are within the scope of the invention.

This invention will be better understood by reference to the Experimental Details which follow, but those skilled in the art will readily appreciate that the specific experiments detailed are only illustrative of the invention as described more fully in the claims which follow thereafter.

### EXPERIMENTAL DETAILS

### EXAMPLE 1

### LB-100 in Combination with Nivolumab increases Interferon-γRelease in CD19 Chimeric Antigen Receptor T-Cells

Protein phosphatase 2A (PP2A) is a hetero-trimeric ubiquitously expressed serine-threonine phosphatase comprised of three subunits: a 65 kDa scaffold subunit PP2A_{α}, a 50-130 kDa regulatory subunit PP2_{β} and a 36 KDa catalytic subunit, PP2_{c}^{1,2}. LB-100, a potent inhibitor of PP2A, has demonstrated safety, tolerability and anti-tumor activity in a dose escalation Phase I clinical investigation in patients with advanced solid tumors³. Additionally, a short hairpin RNA screen by Zhou and colleagues implicates PP2A as an inhibitor of T-cell proliferation in the tumor microenvironment⁴. More recently, Ho and colleagues demonstrated in that the combination of LB-100 and Nivolumab (anti-PD-1) (Nivo) enhanced T cell proliferation in vitro, and decreased tumor burden in a syngeneic CT26 colon carcinoma xeno-transplant mouse model⁵.

We assessed the effects of LB-100 and Nivo on CD19 CART cell function. Propagated CD19CAR T cells were stimulated with dynal beads coated with antibody against CD19 (FMC63) at 1:1 ratio for five days and cultured under the following conditions: CD19CAR T cell alone, CD19CAR+ 0.5 μM LB-100, CD19CAR+ 1.0 μg/mL Nivo, and CD19CAR with combination of LB-100 and Nivo. Drugs were supplemented into the culture every other day. Functional assay was performed six days after treatment. The cells were then co-cultured with the different CD19+ ALL cells SupB15 and different lymphoma Daudi and Raji cells overnight. Cells were then stained for Interferon-gamma (IFNγ) followed by flow cytometry analysis. Acute myeloid leukemia (AML) cells KG1a were used as negative controls. FACS analysis revealed that treatment with LB-100 increased the percentage of CAR+/CD8+/INFγ+ in Daudi (10.99%), Raji (10.30%) and Sup-B15 (10.14%) cells as compared with media alone (7.28%) or KG1A (6.33%). Additionally, the combination of LB-100 with Nivo further increased percentage of CAR+/CD8+/IFNγ+ in Daudi (13.43%), Raji (15.08%) and SupB15 (14.72%) cells as compared with corresponding treatment media alone (8.44%) or KG1A (5.83%). However, IFNγ+ cells in CD4+CAR+ population remained the same. To test the antitumor activity of the combinatorial therapy in vivo we established an acute lymphoid leukemia (ALL) model by inoculating SupB15-GFP-ffluc cells into NSG mice, 5 days after tumor engraftment, mice were treated with CD19CAR T cells by intravenous injection, along with intraperitoneally injections of LB100 (4 μg/mouse), Nivo (200 μgm/mouse) or LB100+Nivo every other day as combination therapy. Consistent with in vitro findings, we found that there is a modest increase in antitumor activity in the combination group 28 days post treatment. In summary, we have shown that combination of LB100 and Nivo can modestly enhance CAR T cell function through IFNγ secretion of CD8+CAR+ T cells. These data lend support for further investigations into the mechanisms behind the effects of combining LB100 and Nivo with CAR T cell therapy.

### REFERENCES

1. Xu Y, Xing Y, Chen Y, Chao Y, Lin Z, Fan E, Yu J W, Strack S, Jeffrey P D, Shi Y. (2006) Structure of the protein phosphatase 2A holoenzyme. Cell. 127(6):1239-1251.
2. Cohen P. (1989) The structure and regulation of protein phosphatases. Annu Rev Biochem. 58:453-508.
3. Chung V, Mansfield A S, Braiteh F, Richards D, Durivage H, Ungerleider R S, Johnson F, Kovach J S. (2017) Safety, Tolerability, and Preliminary Activity of LB-100, an Inhibitor of Protein Phosphatase 2A, in Patients with Relapsed Solid Tumors: An Open-Label, Dose Escalation, First-in-Human, Phase I Trial. Clin Cancer Res. 23(13):3277-3284.
4. Zhou P, Shaffer D R, Alvarez Arias D A, Nakazaki Y, Pos W, Torres A J, Cremasco V, Dougan S K, Cowley G S, Elpek K, Brogdon J, Lamb J, Turley S J, Ploegh H L, Root D E, Love J C, Dranoff G, Hacohen N, Cantor H, Wucherpfennig K W. (2014) In vivo discovery of immunotherapy targets in the tumour microenvironment. Nature. 506(7486):52-57.
5. Ho W S, Wang H, Kovach J S, Lu R, Zhuang Z. Protein phosphatase 2A inhibition with a novel small molecule inhibitor, LB-100, achieves durable immune-mediated antitumor activity when combined with PD-1 blockade in a preclinical model Cancer Research July 2017, [AACR abstract LB-193]. Proceedings: AACR Annual Meeting 2017; 77 (13 Supplement).

1. A method comprising:
(a) providing a population of human T cells harboring a nucleic acid molecule encoding a chimeric antigen receptor; and
(b) culturing the population of human T cells for at least one day in a culture medium comprising one or more exogenously added cytokines and a compound that inhibits the activity of human PP2A.

2. The method of claim 1, wherein the PP2A inhibitor has the structure: 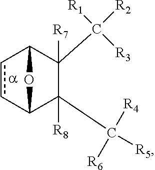
wherein:
bond α is present or absent;
R₁ and R₂ together are ═O;
R₃ is OH, O⁻, OR₉, O(CH₂)₁₋₆R₉, SH, S⁻, or SR₉, wherein R₉ is H, alkyl, alkenyl, alkynyl or aryl;
R₄ is 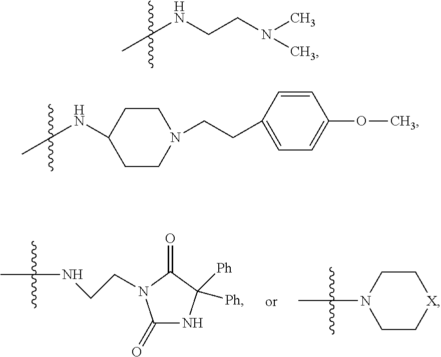
where X is O, S, NR₁₀, N⁺HR₁₀ or N⁺R₁₀R₁₀, where each R₁₀ is independently H, alkyl, alkenyl, alkynyl, aryl, 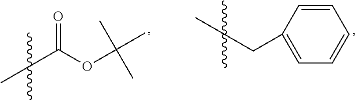
—CH₂CN, —CH₂CO₂R₁₁, or —CH₂COR₁₁, wherein each R₁₁ is independently H, alkyl, alkenyl or alkynyl;
R₅ and R₆ taken together are ═O;
R₇ and R₈ are each H,
or a salt, zwitterion, or ester thereof.

3. The method of claim 2, wherein the PP2A inhibitor has the structure:

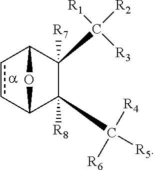

4-5. (canceled)

6. The method of claim 1, wherein the PP2A inhibitor has the structure: 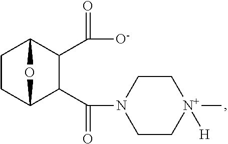 [SMILES: C[NH+]1CCN(C(=O)C2C(C(=O)[O-])[C@H]3CC[C@@H]2O3)CC1]
or a salt or ester thereof.

7. The method of claim 1, wherein the exogenously added cytokines are selected from the group consisting of IL-2, IL-15, IL-7 and IL-21.

8. The method of claim 1, wherein exogenously added IL-2 is present at a concentration of less than 50 U/ml.

9. The method of claim 1, wherein the population of human T cells is cultured for at least 5 days in the culture medium.

10. The method of claim 1 or claim 2, wherein exogenously added IL-15 is present at a concentration of at least 10 ng/ml.

11. The method of claim 1, wherein the population of T cells comprises tumor infiltrating lymphocytes.

12. The method of claim 1, wherein the culture media comprises exogenously added IL-2 at a concentration of less than 10 U/ml.

13. The method of claim 1, wherein the culture media comprises exogenously added IL-2 at a concentration of less than 1 U/ml.

14. The method of claim 1,wherein the culture medium comprises exogenously added IL-7 at concentration of less than 5 ng/ml.

15. The method of claim 1, wherein the culture medium comprises no exogenously added IL-7.

16. The method of claim 1, wherein the culture medium comprises no exogenously added IL-21.

17. The method of claim 1, wherein the culture medium comprises no exogenously added IL-2.

18-19. (canceled)

20. The method of claim 1, wherein the population of cells is cultured for a period of time sufficient to expand the population less than 100-fold.

21-29. (canceled)

30. A method for treating a cancer patient that is being administered recombinant T cells targeted to a tumor cell antigen, comprising administering a compound having the structure: 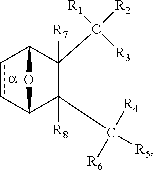
wherein:
bond α is present or absent;
R₁ and R₂ together are ═O;
R₃ is OH, O⁻, OR₉, O(CH₂)₁₋₆R₉, SH, S⁻, or SR₉, wherein R9 is H, alkyl, alkenyl, alkynyl or aryl;
R₄ is 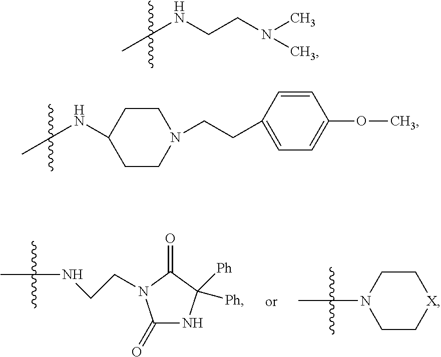
where X is O, S, NR₁₀, N⁺HR₁₀ or N⁺R₁₀R₁₀, where each R₁₀ is independently H, alkyl, alkenyl, alkynyl, aryl, 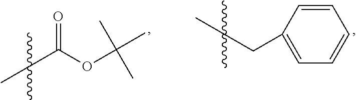
—CH₂CN, —CH₂CO₂R₁₁, or —CH₂COR₁₁, wherein each R₁₁ is independently H, alkyl, alkenyl or alkynyl;
R₅ and R₆ taken together are ═O;
R₇ and R₈ are each H,
or a salt, zwitterion, or ester thereof.

31-36. (canceled)

37. A method comprising:
(c) providing a population of human T cells harboring a nucleic acid molecule encoding a chimeric antigen receptor;
(d) culturing the population of human T cells for at least one day in a culture medium comprising one or more exogenously added cytokines and a compound that inhibits the activity of human PP2A to provide an expanded population of T cells
(e) isolating T cells from the expanded population of T cells; and
(f) administering the isolated T cells to a patient.

38-45. (canceled)

46. A method of enhancing the function of CAR T cells comprising administering to the CAR T cells a PP2A inhibitor so as to thereby enhance the function of CAR T cells.

47. A method of enhancing the function of CAR T cells in a subject afflicted with cancer and undergoing CAR T cell therapy comprising administering to the subject a PP2A inhibitor so as to thereby enhance the function of CAR T cells in the subject.

48-63. (canceled)


SEQUENCE LISTING (5 sequences)

SEQ ID NO: 1 - protein, 41 aa, Homo sapiens
RSKRSRLLHSDYMNMTPRRPGPTRKHYQPYAPPRDFAAYRS

SEQ ID NO: 2 - protein, 41 aa, Artificial Sequence
  Synthetic peptide
RSKRSRGGHSDYMNMTPRRPGPTRKHYQPYAPPRDFAAYRS

SEQ ID NO: 3 - protein, 42 aa, Homo sapiens
KRGRKKLLYIFKQPFMRPVQTTQEEDGCSCRFPEEEEGGCEL

SEQ ID NO: 4 - protein, 112 aa, Homo sapiens
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 5 - protein, 42 aa, Homo sapiens
ALYLLRRDQRLPPDAHKPPGGGSFRTPIQEEQADAHSTLAKI